(19) United States
(12) Patent Application Publication
(10) Pub. No.: US 2003/0199980 A1
(43) Pub. Date: Oct. 23, 2003

(54) Vertebral column implant

(76) Inventor: Uwe Siedler, Alzenau (DE)

Correspondence Address: Friedrich Kueffner, Suite 910, 317 Madison Avenue, New York, NY, 10017, US

(21) Appl. No.: 10/417,968

(22) Filed: Apr. 17, 2003

(30) Foreign Application Priority Data
Apr. 23, 2002 (DE) 102 18 093.8

(51) Int. Cl. A61F 2/44

(52) U.S. Cl. 623/17.11; 606/61

(57) ABSTRACT

A vertebral column implant with two implant elements which each have an anchoring side that faces an adjacent vertebra in the inserted state, wherein each anchoring side has a toothing to ensure against displacement in all directions, and wherein the implant elements can be secured in their relative positions to each other. The vertebral column implant includes implant elements which can be stacked in the manner of a modular system until the desired implant height is reached and which can be secured relative to each other by means of a plug-type connection.

### BACKGROUND OF THE INVENTION

1. Field of the Invention

The present invention relates to an vertebral column implant with two implant elements which each have an anchoring side that faces an adjacent vertebra in the inserted state, wherein each anchoring side has a toothing to ensure against displacement in all directions, and wherein the implant elements can be secured in their relative positions to each other.

2.Description of the Related Art

In a vertebral column implant of the above-described type disclosed in DE 199 02 481 A, two implant elements are provided which form the implant, wherein the implant elements are guided so as to be axially displaceable relative to each other, and wherein the implant elements can be clamped relative to each other in the desired relative position. For this purpose, one of the two elements is composed of segments which extend parallel to the axis, wherein the segments are bendable in the radial direction and are clampable relative to each other by means of a tightening screw acting transversely of the axial direction of the elements.

This prior art implant is cumbersome to manufacture and difficult to manipulate during use.

### SUMMARY OF THE INVENTION

It is the primary object of the present invention to further develop the vertebral column implant in such a way that a vertebral column implant can be provided which in a particularly simple manner can be adapted in its dimensions to the given requirements.

In accordance with the present invention, the vertebral column implant includes implant elements which can be stacked in the manner of a modular system until the desired implant height is reached and which can be secured relative to each other by means of a plug-type connection.

As a result of the configuration according to the present invention, a vertebral column implant can be provided which is dimensioned in accordance with the given requirements, which is stable, and which can be subjected to high loads even when the height of the implant is high. The height of the vertebral column implant depends on the number of implant elements which are stacked one on top of the other, and also on the respective thickness of the individual elements.

In accordance with a particularly useful feature with respect to a secure connection of the individual implant elements, the plug-type connection serving for securing the successive implant elements relative to each other is formed by tongues which protrude from the implant elements parallel to the axis thereof, wherein each tongue includes a transversely extending holding edge which can be engaged by a support edge of the adjacent implant element.

In accordance with an advantageous feature, the implant elements have at least one bore through which the support edge can be engaged by the holding edge of the tongue of the adjacent implant element. In this manner, it is ensured that the tongues are located in the interior of the stack of successive implant elements so as to be protected against external influences. In addition, this advantageously makes it possible to form the support edge to be engaged by the holding edge of the tongue of the adjacent implant element by the rim of the preferably central bore of the adjacent implant element, which simplifies the construction of the vertebral column implant.

It has been found very advantageous with respect to the construction of the implant that the two outer implant elements are connected through the plug-type connection tongues to the respectively adjacent implant element, and that a base element is provided between the outer elements, wherein the base element only has support edges for engaging the holding edges of the tongues of the adjacent implant elements which are inserted from both sides into the base element.

In accordance with a particularly simple embodiment, four implant elements are provided, i.e., the two outer elements which each have an anchoring side facing adjacent vertebrae, and an intermediate implant element formed by a central base element with the two support edges, and at least one implant element serving as a connecting element between one of the outer two implant elements and the central base element.

In accordance with a particularly advantageous embodiment, those implant elements which are provided with support edges for engaging the holding edges of the inserted tongues have transverse bores extending to the axial bores, wherein the tongues ensuring the locking connection between successive implant elements can be laterally elastically deformed for releasing the locking connection by applying a load through the transverse bores. Used for this purpose are pliers having projections whose dimensions are adjusted to the location and size of the transverse bores, wherein the pliers are used for transversely acting on the tongues through the transverse bores for releasing the locking connection as necessary in a very simple manner.

In accordance with another useful further development of the vertebral column implant, the implant elements are constructed in the form of parallelepipeds, wherein each implant element has on one of its edges on the side facing the adjacent implant element in the direction toward the base element a recess in which a projection provided on an adjacent implant element engages. This configuration ensures that for forming the finished vertebral column implant always matching parallelepiped sides rest against each other. The recess is particularly advantageously formed by an inclined surface which interacts with a projection in the form of a prismatic ledge provided at the adjacent implant element.

The various features of novelty which characterize the invention are pointed out with particularity in the claims annexed to and forming a part of the disclosure. For a better understanding of the invention, its operating advantages, specific objects attained by its use, reference should be had to the drawing and descriptive matter in which there are illustrated and described preferred embodiments of the invention.

### BRIEF DESCRIPTION OF THE DRAWING

In the drawing:

FIG. 1 is a perspective exploded view of a vertebral column implant composed of five elements;

FIG. 2a is a front view of a first outer implant element;

FIG. 2b is a side view thereof;

FIG. 2c is a top view thereof;

FIG. 2d is a rear view thereof, and FIG. 2e is a sectional view along sectional line II-II of FIG. 2a;

FIG. 3a is a front view of a central base element;

FIG. 3b is a side view thereof;

FIG. 3c is a top view thereof;

FIG. 3d is a rear view thereof;

FIG. 3e is a sectional view along sectional line III-III of FIG. 3a;

FIG. 3f is a sectional view, on a larger scale, of a detail of FIG. 3e;

FIG. 4a is a front view of a first intermediate implant element;

FIG. 4b is a side view thereof;

FIG. 4c is a top view thereof;

FIG. 4d is a rear view thereof;

FIG. 4e is a sectional view, on a larger scale, along sectional line IV-IV of FIG. 4a;

FIG. 5a is a front view of a second intermediate implant element;

FIG. 5b is a side view thereof;

FIG. 6a is a front view of a second outer implant element;

FIG. 6b is a side view thereof;

FIG. 6c is a top view thereof; and

FIG. 6d is a rear view thereof.

### DETAILED DESCRIPTION OF THE INVENTION

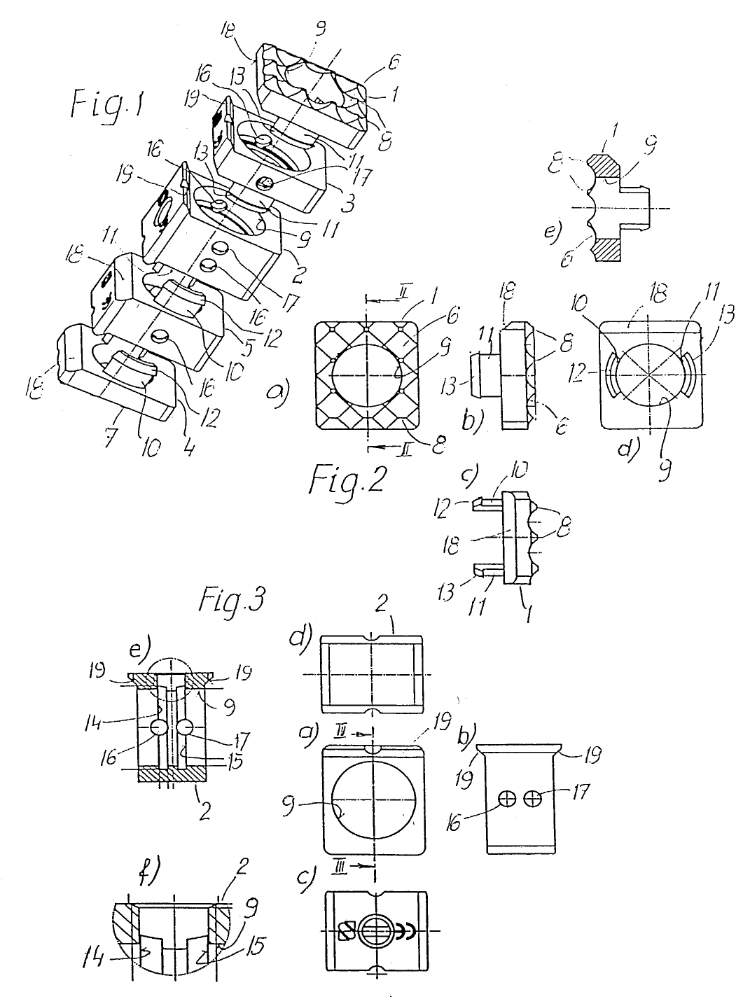

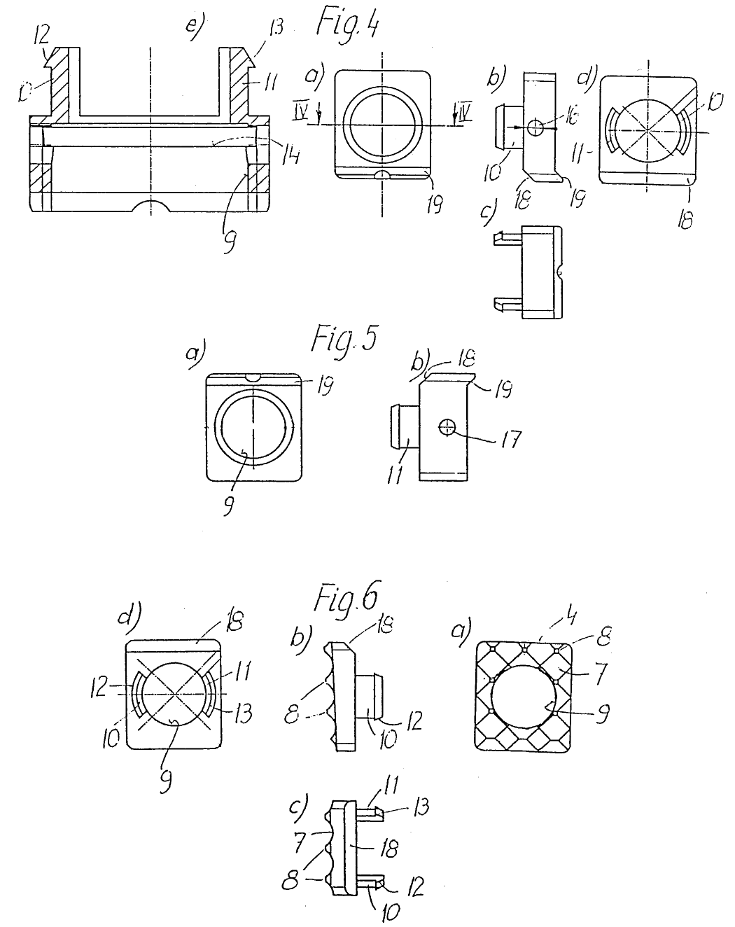

As illustrated in FIG. 1, the preferred embodiment of the vertebral column implant according to the present invention is composed of five implant elements, i.e., a first outer implant element 1, a central base element 2, an implant element 3 located between the first outer implant element 1 and the central base element 2, a second outer implant element 4 and an implant element 5 located between the central base element 2 and the second outer implant element 4. These five implant elements can be securely connected to each other and, in the connected state form the vertebral column implant according to the present invention. The two outer implant elements 1 and 4 each have an anchoring side 6 and 7 facing an adjacent vertebra, wherein the anchoring sides 6 and 7 are provided with recesses with side surfaces which include an angle of about 90 degrees with each other so as to form peaks 8, as illustrated in FIGS. 2 and 6. These peaks 8 form a toothing which ensures that a displacement of the vertebral column implant is prevented toward all sides. On their sides facing away from the anchoring side 6 and 7, the two outer implant elements 1 and 4 are each provided tongues 10, 11 which extend parallel to the axis along the wall of a central bore 9. These tongues 10, 11 have holding edges 12, 13 which extend transversely at the outer ends of the tongues; in the connected state, the holding edges 12, 13 engage behind or grasp a support edge in the area of the bore of the adjacent implant element. Consequently, these tongues 10, 11 form a plug-type connection through which the implant elements 1 through 5 can be connected to each other to form a stable vertebral column implant.

Of course, instead of a single central bore 9, it is also possible to provide two eccentric bores, wherein one of the tongues can be inserted into each of the bores.

As can be seen particularly in FIG. 1 of the drawing, the two outer implant elements 1 and 4 are each connected through the plug-type connecting tongues 10, 11 to the respectively adjacent implant element. The latter, in turn, is connected through analogous tongues to an implant element formed by an intermediate base element 2 without tongues which, as illustrated on a larger scale in FIG. 3f, has in its middle zone two support edges 14, 15 for each of the two holding edges 12, 13 of the tongues 10, 11 inserted into the bores 9.

FIG. 1 as well as FIGS. 3b and 3e clearly show that transverse bores 16, 17 extending to the axial bores 9 are provided in the areas of the holding edges 12, 13 of the tongues 10, 11 which are in the locked-in position. The tongues which ensure the locked connection of successive implant elements can be elastically deformed for releasing the locked connection by applying a load on the tongues through the transverse bores. Used for this purpose may be pliers, not shown in detail, which are provided with two fingers directed toward each other.

As can be further seen in the drawing, the implant elements 1 through 5 each are constructed in the form of a parallelepiped. A recess in the form of an inclined surface 18 is provided on one of the edges of the sides of the implant elements 1 and 3 to 5 facing the base element 2. A projection 19 in the form of a prismatic ledge comes into contact with the inclined surface 18 of an adjacent implant element. The interaction of inclined surface 18 and projection 19 ensures that the implant elements automatically assume their optimum positions.

While FIG. 2 illustrates the implant element 1 having the outer anchoring side 6 as having a uniform thickness, FIG. 6 shows the implant elements 4 having the outer anchoring side 7 as having a non-uniform thickness. Rather, the implant element 4 has a thickness which slightly increases from one outer side to the other outer side in which the inclined surface 18 is provided, so that the vertebral column implant combined into a unit does not have anchoring sides 6, 7 which extend parallel to each other.

As is apparent from the dimensions of the drawing, if the implant elements have a thickness in the range of 3 to 8 mm, vertebral column implants can be formed of different heights in a range of preferably 20 to 50 mm. Of course, it is not necessary that always five elements are combined to form an implant. Rather, the modular system makes it possible that the outer implant element 1 is followed directly by the intermediate base element 2, which, in turn, may be followed by the second intermediate implant element 5 and then the second outer implant element 4.

While specific embodiments of the invention have been shown and described in detail to illustrate the inventive principles, it will be understood that the invention may be embodied otherwise without departing from such principles.

I claim:

1. A vertebral column implant comprising at least two implant elements, wherein each of the two implant elements has an anchoring side adapted to face an adjacent vertebra, wherein the anchoring sides are provided with toothings for preventing displacement of the elements in all directions, wherein the implant elements are of modular construction so as to be stackable until a desired implant height has been reached, and wherein the implant elements are secured relative to each other by a plug-type connection.

2. The vertebral column implant according to claim 1, wherein the plug-type connection is comprised of a tongue protruding from an implant element parallel to an axis of the implant, wherein each tongue comprises a transversely extending holding edge, and wherein each holding edge engages a support edge of an adjacent implant element.

3. The vertebral column implant according to claim 2, wherein each implant element comprises at least one bore, and wherein the support edge is engaged by the holding edge of the tongue of the adjacent implant element extending through the bore.

4. The vertebral column implant according to claim 3, wherein the support edge is formed by a rim of the bore of an implant element which is configured to be engaged by the holding edge of the tongue of an adjacent implant element.

5. The vertebral column implant according to claim 2, comprising a base element connected through the plug-type connection to two outer implant elements, wherein the base element has two support edges configured to be engaged by tongues of the two outer implant elements extending form both sides into the bore of the base element.

6. The vertebral column implant according to claim 5, further comprising a fourth implant element arranged between the base element and one of the outer implant elements.

7. The vertebral column implant according to claim 6, wherein each implant element provided with support edges for engaging the holding edges of inserted tongues has a transverse bore extending to the axial bore, whereby the tongues can be elastically deformed for releasing the plug-type connection by applying a load through the transverse bores.

8. The vertebral column implant according to claim 5, wherein the implant elements are comprised of parallelepipeds, and wherein the implant elements have on sides facing the base element a recess at one of the edges thereof, wherein a projection of an adjacent implant element engages the recess.

9. The vertebral column implant according to claim 8, wherein the recess is an inclined surface and the projection is a prismatic ledge interacting with the inclined surface.
